(19) United States
(12) Patent Application Publication
(10) Pub. No.: US 2009/0198286 A1
(43) Pub. Date: Aug. 6, 2009

(54) BONE FRACTURE FIXATION SYSTEM

(75) Inventors: Antony Lozier, Warsaw, IN (US); Oludele O. Popoola, Granger, IN (US); James J. Mason, Granger, IN (US); Micah Forstein, Warsaw, IN (US)

Correspondence Address: LANDO & ANASTASI, LLP, ONE MAIN STREET, SUITE 1100, CAMBRIDGE, MA, 02142, US

(73) Assignee: ZIMMER, INC., Warsaw, IN (US)

(21) Appl. No.: 12/025,819

(22) Filed: Feb. 5, 2008

(51) Int. Cl. A61B 17/58; A61B 17/56

(52) U.S. Cl. 606/298; 606/280; 606/301

(57) ABSTRACT

A bone fracture fixation system comprising a metallic bone plate having a first composition comprising titanium or a titanium alloy and an opening for receiving a metallic fastener that has a second composition comprising titanium, a titanium alloy, or a stainless steel, and is sized to be received in the opening, and a cold-sprayed metallic coating either within the opening or on the metallic fastener is provided. The cold-sprayed metallic coating comprises a biocompatible metallic material having a third composition different than the first and second compositions. When the metallic fastener is inserted into the opening to stabilize a bone fracture, the cold-sprayed metallic coating may substantially prevent bonding or one or more types of corrosion between the metallic fastener and the metallic bone plate. In another embodiment, the cold-sprayed metallic coating comprises at least one of a cobalt-chrome alloy, gold, a gold alloy, silver or a silver alloy.

### FIELD OF THE INVENTION

This invention relates to bone fracture fixation and, in particular, to a bone fracture fixation system having a coating separating the metals of the system components to prevent undesirable interactions therebetween.

### BACKGROUND OF THE INVENTION

A broken or fractured bone is a serious traumatic injury that can render that portion of the body useless or at least difficult to use, and is usually associated with significant pain. To treat bone fractures, a practitioner may first reduce the bone fragments. As is known in the art, reduction is the process of restoring the parts of the broken bone to their original positions, i.e. normal alignment. When the broken bones are properly aligned, they will knit back together. However, for bone knitting to occur, the bone fragments must remain both aligned and in intimate contact with one another. To that end, the bone fragments must be immobilized. In addition to immobilization, in order for a patient to resume somewhat normal activities, loads must, at least in part, bypass the fracture, particularly loads that cause tensile stress formation at the fracture location.

One familiar method of immobilization and load transmission is via an external fixation device. Traditionally, external fixation devices include plaster or resin casts encased around the injured extremity. The purpose of the external fixation device is to stabilize the position of a fracture so that the bone can mend and allow the patient to use the injured limb. Use of external fixation devices, however, is limited and may depend upon the location of the broken bone, the patient, and other factors.

Alternatively, internal fixation devices are becoming more prevalent. They have found a greater variety of uses since they are not as limited as the external fixation devices. Like external fixation devices, the goal of internal bone fracture fixation is to allow early, pain-free movement and use of the broken bone while maintaining the fracture surfaces in contact so that they heal properly. However, unlike external fixation devices, internal devices do not inhibit either bone vascularity or motion because, for example, they are not as heavy and bulky as external fixation devices. Treatment of bone fractures may, therefore, incorporate reduction followed by installation of an internal fixation device.

Internal fixation devices include, for example, attachment of plates to the outer surface of the bone with at least one fastener. The plates are typically made from titanium, or titanium alloys, and the fasteners are typically made from titanium, titanium alloys, or stainless steel alloys. Usually the fasteners are positioned on each side of the bone fracture to immobilize the adjacent fracture surfaces so that the bone can mend itself, yet allow limited loading of the bone. Any internal fixation device should prevent extreme tensile stress but permit compressive stress at the fracture site.

Internal fixation devices have their own unique drawbacks. For example, internal fixation devices make the bone vulnerable to infection, and the devices may loosen or extrude due to a lack of chemical or biomechanical compatibility and/or incomplete cellular ingrowth. Should any of these occur, it may necessitate removal of the internal fixation device. However, in the case where the internal fixation device comprises a metallic plate held in position by a metallic fastener, the metallic fastener and metallic plate may have, subsequent to installation, spontaneously bonded together due to one of a number of spontaneous bonding mechanisms.

Cold welding is one phenomenon that is characterized by the bonding of metallic parts that are in intimate contact. Usually, cold welding occurs between two similar metals, such as two titanium surfaces. In any case, where the components of an internal fixation device subsequently bond together, the surgeon may have extreme difficultly disengaging one component from the other, such as disengaging a bone screw from within an opening in a bone plate. The bonding may prevent the separation of the components and therefore, may prevent the components from being removed from the patient. Unfortunately, in this situation, the patient suffers from the invasive procedure to remove the internal fixation device and may be unnecessarily injured by the surgeon's efforts to remove the device. Consequently, the patient's initial treatment may complicate, rather than enable, the patient's recovery.

Another potential problem is corrosion of one or more of the components, such as fretting corrosion and/or galvanic corrosion, which can occur between two dissimilar metals, for example, between titanium and stainless steel. When the internal fixation device includes a titanium plate and stainless steel fasteners, galvanic corrosion and/or fretting corrosion may cause the device to fail, and my introduce infection into the bone area being treated. For biocompatibility, it is important that corrosion be avoided or limited.

There is thus a need for an internal bone fixation system where the components do not subsequently bond together or excessively corrode.

### SUMMARY OF THE INVENTION

The present invention provides a bone fracture fixation system comprising (i) a metallic bone plate having a first composition comprising titanium or a titanium alloy and an opening for receiving a fastener, (ii) a metallic fastener, having a second composition comprising titanium, a titanium alloy, or a stainless steel, is sized to be received in the opening, and (iii) a cold-sprayed metallic coating either within the opening or on the metallic fastener. The cold-sprayed metallic coating comprises a biocompatible metallic material having a third composition that is different than the first and second compositions. When the metallic fastener is inserted into the opening of the metallic bone plate to stabilize a bone fracture, the cold-sprayed metallic coating substantially prevents bonding and corrosion between the metallic fastener and the metallic bone plate.

In another embodiment, the metallic bone plate has an opening for receiving the fastener. In addition, the metallic fastener has a head sized to be positioned in the opening and a shaft for inserting through the opening into bone. The cold-sprayed metallic coating comprises at least one of a cobalt-chrome alloy, gold, a gold alloy, silver or a silver alloy, wherein the cold-sprayed metallic coating resides on the head of the metallic fastener. When the metallic fastener is inserted into the opening and engages the metallic bone plate to stabilize a bone fracture, the cold-sprayed metallic coating substantially prevents bonding of the titanium or titanium alloy of the metallic fastener to the titanium or titanium alloy of the metallic bone plate.

In another embodiment, the metallic fastener comprises a stainless steel, and the cold-sprayed metallic coating comprises a metal or alloy having an electrical resistance greater than the stainless steel of the metallic fastener. The cold-sprayed metallic coating resides on the head of the metallic fastener such that when the metallic fastener is inserted into the opening in engagement with the metallic bone plate to stabilize a bone fracture, the cold-sprayed metallic coating provides a barrier to electrical contact between the metallic fastener and the metallic bone plate to thereby substantially prevent galvanic corrosion of the metallic fastener.

In accordance with another aspect of the invention, a method for immobilizing a fractured bone is provided. The method comprises placing a metallic bone plate having an opening for receiving a fastener proximate to the bone. The metallic bone plate has a first composition comprising titanium or a titanium alloy. Subsequent to placing the metallic bone plate, inserting a metallic fastener through the opening and into the bone to secure the metallic bone plate to the bone. The metallic fastener has a second composition comprising titanium, a titanium alloy or stainless steel. A cold-sprayed coating comprising a third composition different than the first and second compositions resides either within the opening or on the metallic fastener and is forcibly engaged between the metallic fastener and the metallic bone plate.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIG. 1 depicts a cross-sectional view of a fractured bone and one embodiment of the bone fracture fixation system secured to the bone;

FIG. 1A is an enlarged view of the encircled area 1A of FIG. 1 illustrating a metallic bone plate, a metallic fastener, and a cold-sprayed metallic coating; and

FIG. 2 shows an exemplary cold-spray coating process for depositing the cold-sprayed metallic coating.

### DETAILED DESCRIPTION

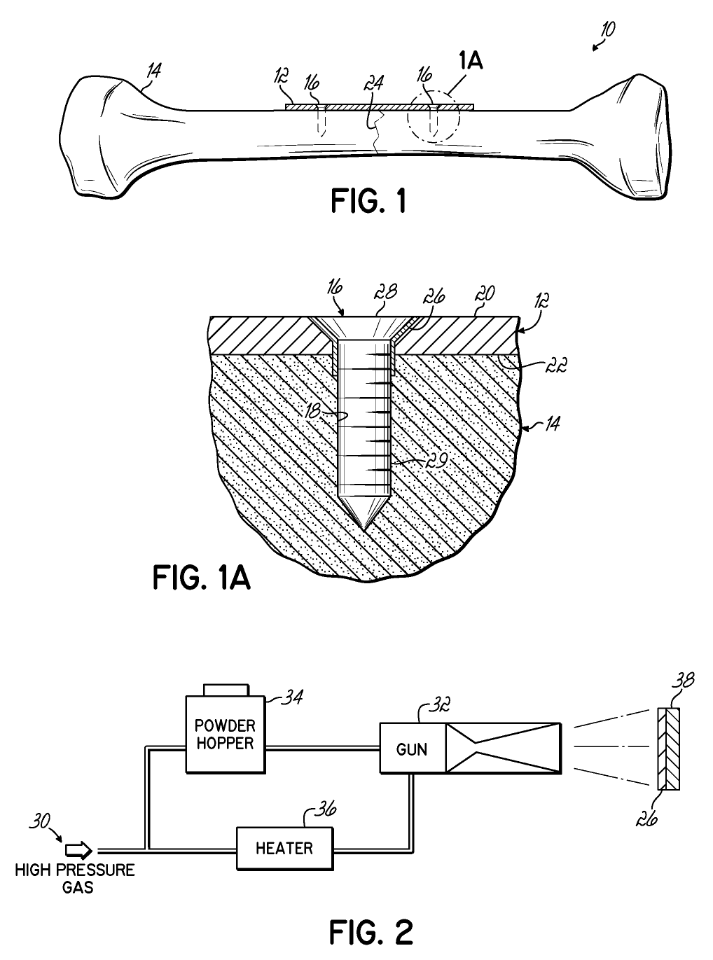

In accordance with the invention and with reference to FIGS. 1 and 1A, an exemplary bone fracture fixation system 10 comprises a metallic bone plate 12 attached to a bone 14 with metallic fasteners 16. The metallic bone plate 12 has at least one opening 18 (best illustrated in FIG. 1A) passing between two opposing surfaces 20, 22 of the metallic bone plate 12. The metallic fastener 16 is positioned through the opening 18 and secured into the bone 14. In the embodiment shown in FIG. 1, the metallic bone plate 12 spans a fracture 24 in the bone 14 with metallic fasteners 16 in openings 18 on each side of the fracture 24. As shown best in FIG. 1A, a cold-sprayed metallic coating 26 is positioned between the metallic fastener 16 and the metallic bone plate 12. One skilled in the art will observe that the cold-sprayed metallic coating 26 may be on the metallic bone plate 12, particularly on the surfaces of the opening 18 between the metallic fastener 16 and the metallic bone plate 12 or on the surfaces of the metallic fastener 16 between the metallic fastener 16 and the metallic bone plate 12.

The metallic bone plate 12 may comprise titanium or a titanium alloy (e.g., Ti-6Al-4V), as is common in the art of internal bone fixation. The metallic fastener 16 may be made of the same or similar biocompatible metal or alloy thereof, i.e. comprises titanium or a titanium alloy, or may be made of a stainless steel (an iron-based alloy with chrome and nickel as the primary alloying elements) (e.g. 316L, which has a composition of: Fe, <0.03% C, 16-18.5% Cr, 10-14% Ni, 2-3% Mo, <2% Mn, <1% Si, <0.045% P, <0.03% S). Stainless steel fasteners have the advantage of increased strength compared to titanium fasteners, while titanium fasteners have the advantage of increase resistance to corrosion, such that either may be desirable. In accordance with the invention, the cold-sprayed metallic coating 26 comprises any biocompatible suitable metal or alloy thereof that is different from titanium and different from stainless steel, e.g., is essentially free of Ti and stainless steel. The metallic composition of the cold-sprayed metallic fastener 16 may be, for example, a cobalt-chrome alloy, tantalum, a tantalum alloy, gold, a gold alloy, silver, or a silver alloy. In one embodiment, the cold-sprayed metallic coating 26 comprises less than 0.1 wt. % titanium. In another embodiment, the cold-sprayed metallic coating 26 is free of any intentional addition of Ti, i.e., only a trace quantity as an impurity from a raw material is contemplated.

One skilled in the art will observe that proper selection of the composition of the cold-sprayed metallic coating 26, the metallic fastener 16, and the metallic bone plate 12 will prevent bonding of the metallic bone plate 12 to the metallic fastener 16 and will prevent or reduce corrosion of one or both of the metallic bone plate 12 and the metallic fastener 16, while allowing the practitioner to select the most appropriate metals for the bone fraction fixation system. Thus, the cold-sprayed metallic coating 26 will facilitate subsequent separation of the plate and fastener components 12, 16 and thus their removal from the bone 14 by normal means, and will help maintain the integrity of the plate and fastener components 12, 16 under corrosive conditions. In other words, the cold-sprayed metallic coating 26 prevents the metallic fastener 16 from bonding to the metallic bone plate 12, and prevents or reduces degradation and/or failure of the plate and fastener components 12, 16 by a corrosion mechanism.

By way of example and not limitation, the metallic bone plate 12 may be made of titanium and the metallic fastener 16 may be made of stainless steel, with the cold-sprayed metallic coating being a cobalt-chrome alloy, tantalum, a tantalum alloy, gold, a gold alloy, silver or a silver alloy. In a further example, the cold-sprayed metallic coating 26 may be essentially free of titanium. In another example, the metallic bone plate 12 may be made of titanium or an alloy thereof and the metallic fastener 16 may also be made of titanium or an alloy thereof, with the cold-sprayed metallic coating 26 being a cobalt-chrome alloy, tantalum, a tantalum alloy, gold, a gold alloy, silver or a silver alloy. By way of further example, the cold-sprayed metallic coating 26 may be essentially free of titanium. In each case, no bonding or appreciable corrosion is expected to occur.

With no intent to be bound by theory, one type of bonding is cold or contact welding. Cold welding was first recognized as a general materials phenomenon in the 1940s. It was then discovered that two clean, flat surfaces of same or similar metal would strongly adhere if brought into contact. Cold welding is the result of metallic asperities on opposing surfaces touching and subsequently bonding to one another. A similar type of bonding may occur between two metal components of the same or similar composition with surfaces in contact while implanted in a biological environment, such as a human body. Significant force is required to break this type of bond making it difficult or impossible to separate bonded components. Thus, where both the plate 12 and fasteners 16 comprise titanium or an alloy thereof, the cold-sprayed metallic coating is applied to one of the plate 12 and the fasteners 16 to prevent the like metal-on-like metal contact that can result in cold or contact welding.

In addition to preventing cold or contact welding, proper selection of the metal composition of the metallic fastener 16 or metallic bone plate 12 and the metal composition of the cold-sprayed metallic coating 26 may prevent fretting corrosion and galvanic corrosion. Fretting corrosion refers to corrosion damage between the asperities of two adjacent surfaces. This damage is induced under load and in the presence of repeated relative surface motion. Galvanic corrosion is an electrochemical process in which one metal, acting as an anode, corrodes preferentially when it is in electrical contact with a different type of metal, acting as a cathode, that is substantially more corrosion resistant (i.e., is more noble) and where both metals are in an electrolyte. Bodily fluids may act as an electrolyte. Galvanic corrosion may create debris, which may also then serve as asperities that promote fretting. Thus, material interactions that promote one or more corrosion mechanisms are undesirable, and can be avoided or reduced by selection of an appropriate coating material. For example, reducing or eliminating electrical conduction by coating stainless steel fasteners with a more electrically resistant metal or alloy, i.e., an electrical resistance sufficient to reduce electrical conduction between the metals that can drive a galvanic corrosion mechanism. By way of example, a stainless steel fastener 16 may be coated with a metal or alloy having an electrical resistivity sufficient to restrict current flow between the metallic fastener 16 and the metallic bone plate 12 thereby reducing the galvanic potential. Alternatively, the stainless steel fastener 16 or the metallic bone plate 12 may be entirely encased in another metal, such as a tantalum alloy or a cobalt-chrome-molybdenum alloy to reduce the galvanic potential between the metallic fastener 16 and the metallic bone plate 12. It is within the skill of one in the art to select the coating material based upon the particular metals or alloys comprising the plate 12 and fasteners 16 such that current flow between the metallic fastener 16 and the metallic bone plate 12 is sufficiently low.

One skilled in the art will appreciate that the metallic fastener 16 may be one selected from a variety of commercially available metallic fasteners. By way of example, the metallic fasteners may be cortical bone screws, cancellous bone screws, self-tapping screws, non-self tapping screws, lag screws, nails, or wire.

The metallic bone plate 12 may be, for example, a neutralization plate that is used to protect a screw from torsional, bending, and shearing forces; a compression plate that is used to provide compression at fracture site; a buttress plate used to support bone fragments from shearing forces, or an antiglide plate used to immobilize oblique fractures. Moreover, the metallic bone plate 12 may be a flexible mesh or possibly an intramedullary rod.

In one embodiment, the metallic fastener 16 has a head 28 sized to be positioned in the opening 18, as shown in FIG. 1A. The metallic fastener 16 also has a shaft 29 that passes through the opening 18 to engage the bone 14. Furthermore, the cold-sprayed metallic coating 26 may be formed on a surface of the opening 18 or on a surface of head 28. Therefore, the coating 26 resides between the metal (or alloy thereof) of the metallic bone plate 12 and the metal (or alloy thereof) of the metallic fastener 16, particularly at all surfaces where the metallic fastener 16 is in direct contact with the metallic bone plate 12.

In one embodiment, the cold-sprayed metallic coating 26 is a functionally graded coating. By functionally grading the coating 26, the durability of the coating 26, particularly its resistance to delamination, may be enhanced, for example, by providing a less sharp chemistry interface in the transition zone from the substrate (the plate 12 or the fastener 16) to the coating 26. Composite coatings can be applied consisting of a mixture of metals of different types by using two sprays of the different metals. The ratio of the two metals can be varied during the build-up of the coating. For example, cobalt and chromium can be sprayed simultaneously from separate nozzles and their ratios continuously or discontinuously varied to provide decreasing chromium content from the substrate to the coating surface, or vice-versa, as desired. In another example, titanium may be sprayed onto a titanium fastener and the spray transitioned to cobalt-chromium so as to achieve a gradual change in chemistry from the titanium fastener material to the cobalt-chrome coating. Thus, the composition of the coating 26 may comprise a single metal or alloy thereof, may comprises a graded metallic composition, or may be a layered composite of metals and/or alloys thereof.

The cold-sprayed metallic coating 26 is formed by cold spraying, as is known in the art of metal coatings. As shown in FIG. 2, cold spraying uses a high-pressure gas supply 30 and a spray gun 32. A powder hopper 34 supplies metal powder in a carrier gas to the spray gun 32, and a heater 36 heats the high-pressure gas separately supplied to the spray gun 32. Cold spray processing is known to generate a high velocity gas that accelerates the powder particles to high velocity before they impact a substrate 38. The bond between the particle and the substrate 38 is created by kinetic rather than thermal energy. A powder having the composition desired for the coating 26 is cold sprayed onto the metallic fastener 16 or onto the surface within the opening 18 of the metallic bone plate 12. With cold spray processing, relatively thick coatings of at least about 250 μm (about ⅛ inch) may be sprayed onto the surface within the opening 18 of the metallic bone plate 12 or onto the metallic fastener 16. In one embodiment, the coating 26 is sprayed to a thickness of about 250 μm to about 12.7 mm (about ⅛ inch to about ½ inch). Post-spray machining of the cold-sprayed metallic coating 26 may be used to generate optimum dimensions between the head 28 and the shaft 29 of the metallic fastener 16 and the opening 18 in the metallic bone plate 12. Cold spraying is not limited to spraying a powder of a single composition. As previously described, multiple powders having different compositions may also be deposited, concurrently or sequentially. For example, functionally graded coatings may be sprayed onto the surface of the metallic fastener 16 or the metallic bone plate 12.

In one embodiment, a method for immobilizing a fractured bone comprises first exposing a portion of the fractured bone 14, then placing the metallic bone plate 12 proximate to the bone 14 and securing the metallic bone plate 12 to the bone 14 with a metallic fastener 16. The method may further include machining a hole into the bone 14 prior to inserting the metallic fastener 16. Machining may include drilling with a cannulated drill, tapping the hole to create threads, and then screwing the metallic fastener 16 into the bone 14. By securing the metallic fasteners 16 into the bone 14, the cold-sprayed coating 26 is forcibly engaged between the metallic bone plate 12 and the metallic fastener 16. According to one embodiment, as shown in FIG. 1, two metallic fasteners 16 are used to attach the metallic bone plate 12 to the bone 14. As one skilled in the art will observe, while two metallic fasteners 16 are shown, at least one metallic fastener 16 or a plurality of metallic fasteners 16 may be used to secure the metallic bone plate 12 to the bone 14.

While the present invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method and illustrative examples shown and described. Accordingly, departures may be made from such details without departing from the scope of the general inventive concept.

What is claimed is:

1. A bone fracture fixation system comprising:
a metallic bone plate having a first composition comprising titanium or a titanium alloy and an opening for receiving a fastener,
a metallic fastener having a second composition comprising titanium, a titanium alloy, or a stainless steel, and sized to be received in the opening, and
a cold-sprayed metallic coating either within the opening or on the metallic fastener and comprising a biocompatible metallic material having a third composition that is different than the first and second compositions such that when the metallic fastener is inserted into the opening of the metallic bone plate to stabilize a bone fracture, the cold-sprayed metallic coating substantially prevents bonding and corrosion between the metallic fastener and the metallic bone plate.

2. The bone fracture fixation system of claim 1 wherein the cold-sprayed metallic coating is applied to a portion of the metallic fastener that contacts the metallic bone plate within the opening.

3. The bone fracture fixation system of claim 1 wherein the cold-sprayed metallic coating is applied within the opening of the metallic bone plate.

4. The bone fracture fixation system of claim 1 wherein the metallic fastener has a head and a shaft, and wherein the head forcibly engages the metallic bone plate within the opening when the metallic fastener attaches the metallic bone plate to bone, and wherein the cold-sprayed metallic coating is applied to the head.

5. The bone fracture fixation system of claim 1 wherein the metallic fastener comprises a stainless steel.

6. The bone fracture fixation system of claim 5 wherein the cold-sprayed metallic coating is on the metallic fastener and comprises a cobalt-chrome alloy.

7. The bone fracture fixation system of claim 1 wherein the metallic fastener comprises titanium or a titanium alloy, and the cold-sprayed metallic coating comprises a cobalt-chrome alloy.

8. The bone fracture fixation system of claim 1 wherein the cold-sprayed metallic coating comprises a cobalt-chrome alloy, tantalum, a tantalum alloy, gold, a gold alloy, silver and/or a silver alloy.

9. The bone fracture fixation system of claim 1 wherein the metallic fastener is one of a screw, nail, pin, bolt, or wire.

10. The bone fracture fixation system of claim 1 wherein the cold-sprayed metallic coating is at least approximately 250 μm thick.

11. A bone fracture fixation system comprising:
a metallic bone plate comprising titanium or a titanium alloy and having an opening for receiving a fastener;
a metallic fastener comprising titanium or a titanium alloy, wherein the metallic fastener has a head sized to be positioned in the opening and a shaft for inserting through the opening into bone; and
a cold-sprayed metallic coating comprising at least one of a cobalt-chrome alloy, gold, a gold alloy, silver or a silver alloy, wherein the cold-sprayed metallic coating resides on the head of the metallic fastener such that when the metallic fastener is inserted into the opening in engagement with the metallic bone plate to stabilize a bone fracture, the cold-sprayed metallic coating substantially prevents bonding of the titanium or titanium alloy of the metallic fastener to the titanium or titanium alloy of the metallic bone plate.

12. The bone fracture fixation system of claim 11 wherein the cold-sprayed metallic coating is at least approximately 250 μm thick.

13. The bone fracture fixation system of claim 11 wherein the metallic fastener is a screw, nail, pin, or bolt.

14. A bone fracture fixation system comprising:
a metallic bone plate comprising titanium or a titanium alloy and having an opening for receiving a fastener;
a metallic fastener comprising a stainless steel, wherein the metallic fastener has a head sized to be positioned in the opening and a shaft for inserting through the opening into bone; and
a cold-sprayed metallic coating comprising a metal or alloy having an electrical resistance greater than the stainless steel of the metallic fastener, wherein the cold-sprayed metallic coating resides on the head of the metallic fastener such that when the metallic fastener is inserted into the opening in engagement with the metallic bone plate to stabilize a bone fracture, the cold-sprayed metallic coating provides a barrier to electrical contact between the metallic fastener and the metallic bone plate to thereby substantially prevent galvanic corrosion of the metallic fastener.

15. The bone fracture fixation system of claim 14 wherein the cold-sprayed metallic coating is at least approximately 250 μm thick.

16. The bone fracture fixation system of claim 15 wherein the metallic fastener is a screw, nail, pin, or bolt.

17. A method for immobilizing a fractured bone comprising:
placing a metallic bone plate having an opening for receiving a fastener proximate to the bone, the metallic bone plate having a first composition comprising titanium or a titanium alloy; and
inserting a metallic fastener through the opening and into the bone to secure the metallic bone plate to the bone, the metallic fastener having a second composition comprising titanium, a titanium alloy or stainless steel, and wherein a cold-sprayed coating comprising a third composition different than the first and second compositions resides either within the opening or on the metallic fastener and is forcibly engaged between the metallic fastener and the metallic bone plate.

18. The method of claim 15, further comprising machining a hole into the bone for receiving the metallic fastener prior to inserting the metallic fastener into the bone.

19. The method of claim 17 wherein the cold-sprayed metallic coating resides on a portion of the metallic fastener that contacts the metallic bone plate within the opening.

20. The method of claim 17 wherein the cold-sprayed metallic coating resides on a surface within the opening of the metallic bone plate.

21. The method of claim 17 wherein the metallic fastener has a head and a shaft, and upon inserting, the shaft enters into the bone while the head forcibly engages the metallic bone plate within the opening, and wherein the cold-sprayed metallic coating resides on the head.

22. The method of claim 17 wherein the metallic fastener comprises a stainless steel and the cold-sprayed metallic coating is on the metallic fastener and comprises a cobalt-chrome alloy.

23. The method of claim 17 wherein the metallic fastener comprises titanium or a titanium alloy, and the cold-sprayed metallic coating comprises a cobalt-chrome alloy.

24. The method of claim 17 wherein the cold-sprayed metallic coating comprises a cobalt-chrome alloy, tantalum, a tantalum alloy, gold, a gold alloy, silver and/or a silver alloy.

25. The method of claim 17 wherein the cold-sprayed metallic coating is at least approximately 250 μm thick.
